(19) United States
(12) Patent Application Publication
(10) Pub. No.: US 2020/0275687 A1
(43) Pub. Date: Sep. 3, 2020

(54) EDIBLE MATERIALS AND EDIBLE ELECTRICAL SYSTEMS MADE OF SAME

(71) Applicants: Arizona Board of Regents on Behalf of the University of Arizona, Tucson, AZ (US); Arizona Board of Regents on Behalf of Arizona State University, Scottsdale, AZ (US)

(72) Inventors: Hanqing Jiang, Chandler, AZ (US); Wenwen Xu, Mesa, AZ (US); Haokai Yang, Tempe, AZ (US); Wei Zeng, Mesa, AZ (US); Hongbin Yu, Chandler, AZ (US); Marvin J. Slepian, Tucson, AZ (US)

(21) Appl. No.: 16/612,069

(22) PCT Filed: May 9, 2018
(86) PCT No.: PCT/US2018/031900

§ 371 Date: Nov. 8, 2019

Related U.S. Application Data
(60) Provisional application No. 62/503,665, filed on May 9, 2017.
(60) Provisional application No. 62/516,498, filed on Jun. 7, 2017.

(51) Int. Cl. A23P 20/10; A61B 5/00; A23L 29/294

(52) U.S. Cl. CPC A23P 20/10; A23L 29/294; A61B 5/686

(57) ABSTRACT

Edible electrical systems containing food and/or food-based materials that possess electrical characteristics comparable or substantially equal to those of conventional materials used traditional electronic circuits and configured to include at least one of a capacitor, resistor, inductor, microphone, RF filter, pH monitoring device to collect information representing a living biological tissue from inside a body. A set of materials for use in constructing such edible electrical systems and methods of using such systems for acquiring such information. Edible electrical systems may be configured to be implantable.

### CROSS-REFERENCE TO RELATED APPLICATIONS

This application is a US National Stage of PCT Application No. PCT/US2018/031900 filed on May 9, 2018, which claims benefit of a priority from the US Provisional Application Nos. 62/503,665 filed on May 9, 2017 and 62/516,498 filed on Jun. 7, 2017. The disclosure of each of the above-identified patent applications is incorporated herein by reference.

### TECHNICAL FIELD

This disclosure pertains to electronic devices and components and, in particular, to electronic devices and components formed in whole or in part from food materials, food-like materials, and/or foodstuffs as well as the gamut of food-related materials supporting the idea of fabrication of such electronic devices and components for intended purpose(s) and related method of fabrication.

### BACKGROUND

Advances in material science have led to the evolution of biomedical electronic devices from non-implantable electronic devices, to implantable, to partially biodegradable, and, most recently, to physically transient electronic devices.

Various devices located in association with skin (on the skin, for example) are formatted to detect variables such as heart rate, temperature, and sweat-based body constituents. Implantable systems can be more robust, but they are typically more invasive and present a potential risk of infection, bleeding, and a need for surgical recovery in the event of a malfunction. Some of the implantable devices include implantable cardiac devices, intracranial pressure sensors, and a swallowable capsule-based devices configured to measure temperature, pressure, effectuate imaging and measure acidity level (pH) data to complement diagnostics and local drug delivery.

Devices, in which the degree of biocompatibility is higher are clearly more preferred, as such device are less likely to elicit inflammatory reactions leading, for example, to restenosis in arteries or to tissue scarring in regional implants.

Biodegradable electronic devices discussed thus far include a primary battery and biosensor, and an organic field effect transistor. Notably, completely biodegradable devices still remain a goal, not a reality, primarily due to the lack of biodegradable materials with electrical properties that are comparable to those of available non-degradable metals and insulating materials that are used in conventional passive implantable devices.

Implementations of physically transient electronics have been attempted and include a device that can act as a programmable, non-antibiotic bacteriocide; transient devices that incorporate degradable device components, degradable substrates, and/or degradable encapsulating materials; an implantable, tunable, biodegradable medical device for nerve stimulation within the body of a patient; and ingestible and/or digestible electronic devices for diagnostic and therapeutic applications. It is well recognized that the results of these initial demonstrations had several significant limitations. One of the practical limitations stems from the fact that the prototypes are typically fabricated using materials that are permanent in shape and/or form and/or substance and largely non-degradable, and thus may require physical retrieval (for example, when these devices have already performed their functions and are no longer required, or in the advent of malfunction). Even if the requirement of or need in retrieval of a given physically transient electronic device is reduced or even eliminated, such device still requires invasive measures for implantation, often via surgery, and thus inherits all usual drawbacks associated with surgery (such as high costs and risks associated with complications that may occur during surgery, after surgery, or both. Another limiting aspect of existing physically transient systems is that—in order to or in attempt to enhance the biodegradability of such systems and to limit their toxicity and reduce the adverse effects of using such systems—these systems are fabricated with the use of traditional electronics materials but on the geometrically-reduced scale (for example, on a nanoscale) regime in order to enhance their biodegradability and limit toxicities and adverse events.

Notably, the making of a physically transient electronic device still utilizes traditional microelectronics materials that have limited biocompatibility, are often toxic, and overall not necessarily safe for the human body.

### SUMMARY

Embodiments of the present invention provide a set of materials for use in fabrication of an electrical system. Such set of materials includes any of food, a food-like material, a foodstuff, a food-based material, a synthetic material, and a natural material. In one implementation, the set of materials includes a substantially dry material. A set may additional contain a material selected from a group consisting of carbon, non-toxic metal, and a non-toxic non-metallic inorganic material and/or a material representing an electrically-conducting material, a dielectric material, or a piezoelectric material.

Embodiments of the invention also provide an edible electrical system that includes at least one material from a first set of materials containing food, a food-like material, a foodstuff, a food-based material, a synthetic material, and a natural material. (In one specific case, the first set of materials includes at least one of a plasticizer and sorbitol.) Alternatively or in addition, the edible electrical system includes at least one material from a second set of materials that includes carbon, non-toxic metal, and a non-toxic non-metallic inorganic material while, optionally, a material from at least one of the first and second sets of materials is an electrically-conducting material, a dielectric material, or a piezoelectric material. In any of these cases, the system may include a component configured as at least one of an electrically-insulating member, a dielectric member, an electrically-conducting member and/or a component configured as at least one of a resistor, a capacitor, an inductor, an antenna, an interconnect, a transistor, a diode, a substrate, a wire, and a printed circuit board. (A wire may be twisted, in the system.) In any of these cases, the edible electrical system may also be configured as an implantable system and/or comprise at least one of a microphone and a pH sensor. In any of these implementation, the first set of materials may include one or more of a vegetable, fruit, bread, flour, oil, carbonized cotton candy, hard candy, cotton, carbonized cotton fiber, silk, carbonized silk, bone, tendon, gelatin, grain, sugar, active, charcoal, marshmallow, a component of an egg, and starch. Substantially any embodiment may include at least one component or a group of components that is/are coated with a coating material from the first set of materials (in a specific case of the latter, the coating material is disposed to substantially encapsulate such component or group of components and/or the coating material includes at least one of a gelatin and a sugar paste. A component of the system may be mounted on an edible printed circuit board and, optionally, operationally connected to a microchip electronic circuitry. In a related embodiment, at least two components are connected directly or indirectly to electronically communicate with one another other. Any embodiment of the edible electrical system may additionally be equipped with a power source configured to provide power to at least one of components of the system that is required for proper functioning of such component(s).

Embodiments additionally provide an implantable electrical system that contains at least one of i) a first material from a first set of materials that includes food, a food-like material, a foodstuff, a food-based material, a synthetic material, and a natural material, and ii) a second material from a second set of materials that includes carbon, non-toxic metal, and a non-toxic non-metallic inorganic material The system may additionally include at least one of a) a component configured as at least one of an electrically-insulating member, a dielectric member, an electrically-conducting member; and b) a component configured as at least one of a resistor, a capacitor, an inductor, an antenna, an interconnect, a transistor, a diode, a substrate, a wire, a printed circuit board, and a microchip. In any implementation, the system may be configured for being administered orally, buccally, nasally, rectally, or vaginally. In any implementation, the system may contain a device selected from the group consisting of a pH sensor, a radio frequency (RF) filter, a microphone, a mechanical sensor, a temperature sensor, a pressure sensor, an analyte sensor, a microbial sensor, and a fluid-flow sensor. Substantially any embodiment of the implantable electrical system may be configured for use in food storage or food packaging. In a specific case, a component of the system may be configured to be at least one of edible, biodegradable, and ingestible.

Furthermore, embodiments provide an implantable electrical system that includes at least one of i) a first material from a first set of materials that includes food, a food-like material, a foodstuff, a food-based material, a synthetic material, and a natural material, and ii) a second material from a second set of materials that includes carbon, non-toxic metal, and a non-toxic non-metallic inorganic material, and that, additionally, has at least one of a) a component configured as at least one of an electrically-insulating member, a dielectric member, an electrically-conducting member, and b) a component or device configured as at least one of a resistor, a capacitor, an inductor, an antenna, an interconnect, a transistor, a diode, a substrate, a wire, a printed circuit board, and a microchip. In substantially any case, the implantable electrical system may optionally contain a device selected from the group consisting of a pH sensor, a radio frequency (RF) filter, a microphones a mechanical sensor, a temperature sensor, a pressure sensor, an analyte sensor, a microbial sensor, and a fluid-flow sensor.

### BRIEF DESCRIPTION OF THE DRAWINGS

FIG. 1 contains a schematic diagram of a food pyramid and a table summarizing electrical properties of foods from various food groups.

FIGS. 2A, 2B, 3A, and 3B are graphs representing EDX test results for various food materials.

FIG. 4 is a magnified view of the microscale morphology of carbonized cotton candy.

FIG. 5 is a magnified view of the microscale morphology of cotton

FIG. 6 is a magnified view of the microscale morphology of silk.

FIG. 7 is a table listing electrical conductivity values of carbonized cotton candy, cotton, and silk materials.

FIGS. 8A, 8B show an experimental set up for characterizing the piezoelectric coefficient d₃₃ of materials and schematic of a sample and electrode design, respectively.

FIG. 9 is a schematic illustration of relative conductivity of various different types of edible materials.

FIG. 10 is a magnified view of the microscale morphology of broccoli powder.

FIG. 11 is a magnified cross-sectional view of an edible piezoelectric thin film.

FIGS. 12A, 12B, 12C, 12D are line graphs comparing the piezoelectric performance of commercial lead zirconate titanate (PZT) film (FIG. 12A), with those of films made with broccoli/gelatin (FIG. 12B), cabbage/gelatin (FIG. 12C), and cauliflower/gelatin (FIG. 12D).

FIGS. 13A, 13B illustrate cross-sectional and outside images of an embodiment of the edible capacitor.

FIG. 14 contains plots representing empirically-measure capacitance, of various capacitor embodiments, as a function of frequency.

FIGS. 15A, 15B are SEM and optical images of an embodiment of an edible resistor.

FIGS. 16A, 16B are SEM and optical images of an embodiment of an edible inductor.

FIGS. 17A, 17B contain line plots of resistance test results formed from different composite materials as compared to that formed from active charcoal, as a function of length.

FIGS. 18A, 18B, 18C are line graphs showing the operational performance of inductors with different diameters and lengths.

FIGS. 19A, 19B, 19C present a perspective view, morphology, and a graph of resistance of an embodiment of a wire formed from edible materials.

FIGS. 20A, 20B, 20C illustrate is a perspective view of an embodiment of an antenna and graphs of output voltage of the antenna as a function of input voltage and as a function of time, respectively.

FIGS. 21A, 21B, 21C show components of an edible pH sensor. FIG. 21A is an image of an edible pH sensor containing gold and zinc (II) oxide (Au—ZnO) as working electrodes, an antenna made of gold for wirelessly transmitting signals, and an edible capacitor, on an edible matzoth substrate. FIG. 21B is an expanded view of the electronic components of the edible pH sensor of FIG. 21A. FIG. 1C is a schematic of an electrical construct containing an edible pH sensor and an external device for detecting and measuring signals transmitted from the edible pH sensor.

FIG. 22 provides plots illustrating empirically determined the capacitance and resonance frequency values of the embodiment of the edible pH sensor of FIGS. 21A, 21B, 21C.

FIGS. 23A, 23B illustrate an embodiment of an edible radio frequency filter and simulated and measured frequency responses for a series of values of resistivity, inductance, and capacitance of the constituent components of the filter.

FIG. 24A illustrates a top view of a PCB made of powdered sugar with deposited gold layers.

FIG. 24B is a prospective view of a bread board (cracker/saltine) with a conductor (carbonized and inductor—wound carbonized material).

FIGS. 25A, 25B illustrate an embodiment of an edible piezoelectric microphone and results of its characterization. FIG. 25A: an image of the edible piezoelectric microphone. FIG. 25B: a set up used to characterize the edible piezoelectric microphone.

FIGS. 26A, 26B, 26C, 26D, 26E show voltage waveforms recorded at different frequencies with the embodiment of FIG. 25A, and illustrating fidelity of recorded sound using the embodiment of FIG. 25A.

FIGS. 27A, 27B present the amplitude of input abdominal sound, and amplitude of recorded abdominal sound, respectively, acquired with the embodiment of FIG. 25A.

FIGS. 28A, 28B are schematics of structures used to perform electrical conductivity measurements of materials. FIG. 28A illustrates a “sandwich” structure for electrical conductivity measurement of non-liquid materials. FIG. 28BB shows a sample holder for electrical conductivity measurements for liquid materials.

FIG. 29 is a schematic diagram of a measurement setup for characterization of a materials piezoelectric properties, in terms of d₃₃.

FIGS. 30A, 30B, 30C illustrate theempirically-measured mechanical proporties of different doughs: flour dough, powdered sugar dough, and sweet potato dough, respectively.

FIG. 31 is a line graph of resistance test results of different lengths of wires with coating times of 1 min, 5 mins, and 10 mins.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another.

### DETAILED DESCRIPTION OF THE INVENTION

The persisting limitations caused, in the field of fabrication of microelectronic components, by the lack of materials that can be combined with the biological tissue in a very specific, non-toxic and bio-absorbable fashion begs a question of whether one can realize microelectronic devices that are compatible with the living tissue while leaving very little—if any—trace or impact on the living tissue inside which such devices are disposed.

For example, despite the fact that the gastrointestinal (GI) provides one of the primary interfaces between the environment that is external to the body and the internal milieu and, for that reason provides a tremendous surface area for device residence and monitoring of a wide range of health and disease conditions and states, the GI tract remains a bodily domain that has only been partially explored to date. Embodiments of the present invention address this limitation by providing a set or kit of materials that are fit for fabrication of an implantable and/or edible electrical system that are non-toxic and physiologically safe when used inside the human body and that are alternative and/or complementary to the materials conventionally used for fabrication of electrical or electronic circuits and systems to-date. Embodiments additionally provide electronic devices and components fabricated from such materials. The availability of such set of materials for fabrication of electrical systems in question—at least a portion of which includes food-related materials that have not been utilized for this purposes to-date, as discussed below—and the systems themselves substantially broadens the choice of materials for microelectronic fabrication and expands the applications and uses of so-configured electrical systems to the situations encompassing the characterization of the biological tissue associated with the GI tract and/or other cavities or orifices of the human body.

For the purposes of this disclosure and appended claims, the term “edible” is defined as a thing fit to be positioned or disposed in a GI tract and generally recognized as safe (and whether or not such edible thing possesses any nourishment or sustenance). As far as this term identifies an electrical system, this term defines an electrical system that can be eaten or otherwise taken into the GI tract via alimentation means, for example (such as a nasogastric, NG, tube or gastrostomy tube), or via swallowing, ingestion with fluid, ingestion with a lubricating solution, or actual chewing, mastication and deglutition followed by swallowing. Edible electrical systems include the systems that are configured to resist abrasion, surface damage or compaction, and/or reduction of size while remaining substantially functionally (operationally) intact, swallow-able, and capable of traversing the GI tract. The term “implantable”, on the other hand, is defined as being fit for incorporation into a living biological tissue (for example, into a human body) without any substantially adverse defect such as, for example, poisoning of the biological tissue. As used herein, an edible thing is an implantable thing. As applied to metallic or other inorganic materials, the terms “edible” and “implantable” are defined to imply that—if internally consumed by a human—such materials remain non-toxic, non-poisonous, non-venomous, non-virulent and generally not destructive of the human health.

The term “electrical system” refers to either a single, stand-alone electronic component or a group of such components operably cooperated with one another to form a device structured to operate as intended (and optionally powdered with a power source). A “component” refers to a material or individual piece that is used to fabricate an electrical system. (Examples of individual pieces, components, and/or devices of an edible/implantable electrical system include a transistor, a resistor (e.g. a potentiometer), a capacitor, an inductor, electrodes, an insulator material, a conducting material and or wire/interconnect made from such material, an antenna, and a diode.)

As discussed below, some embodiments of implantable and/or edible electrical systems disclosed below are at least partially biodegradable. Alternatively or in addition, such embodiments are configured to ensure that i) positioning of these embodiments inside a biological tissue substantially lacks (does not require or imply) invasive measures associated with changing a structure of the biological tissue, and/or ii) that these embodiments are either digestible or bioabsorbable such that, upon the completion of the useful operation of such a system disposed inside the biological tissue, at least a portion of the system is dissolved or otherwise absorbed by the biological tissue while the remaining, non-absorbable portion of the system is either removed by the biological system (such as, for example, via excretion) or can indefinitely remain in the biological tissue due to their substantially miniscule size without producing a harmful effect on the biological tissue. Even if the requirement of or need in retrieval of a given physically transient electronic device is reduced or even eliminated, such device still requires invasive measures for implantation, often via surgery, and thus inherits all usual drawbacks associated with surgery

Accordingly, a problem of current inability of the related art to acquire data that represent status of a living biological tissue from inside the tissue without detrimentally affecting the structure and/or the living parameters of the tissue (such as viability, for example) or without leaving a system chosen to perform such measurement inside the tissue to a substantial length of time is solved by devising a measurement methodology with the use of an electrical system located in the middle of the tissue (for example, in a GI tract) that allows the collection of sought-after data at any moment of time that precedes the dissolution/absorption of an food-based component of such system by the living tissue.

### Determination of Sets of Materials

The described electrical systems encompass electronic components and electronic devices. The electronic components are fabricated using one or more of the food materials, processed food materials, food-like materials, foodstuffs, and natural materials, or a combination thereof. Preferably, the electronic components include at least one or more food materials.

As used herein and unless expressly defined otherwise:

The term “food” is used to refer to materials that are ingested to provide satiety or nutritional support for an organism. This term includes, for example, elemental unprocessed or raw foods, components or a finished system suitable for eating as a typical human food. Examples of raw food include bananas or other fruits and raw meat or fish.

Processed food is commonly understood as food that has been processed prior to and/or after cooking. Accordingly, the term “processed food” is defined to refer to a food material that has been subjected to a transformation process to convert it to another material that can also be safely consumed by a human. The transformation process can be physical, chemical, or both. A physical transformation can include cooking, backing, and/or drying, while chemical transformation can include carbonization. Examples of processed food include cheese, lavosh, matzoth, or potato chip.

The term “food-like” refers to materials that are largely systematized from biochemical compounds or other organic constituents, though are not typically utilized for food by the majority of the world population. Examples here are provided by chitosan and alginates.

The term “foodstuff(s)” refers to materials that are systematized or fabricated from source biological materials, such as animals and plants, which may be utilized as foods or are components of foods. Examples here include soy protein or whey.

The term “food-based” broadly refers to food materials, processed food materials, food-like materials, and foodstuffs, as defined above.

“Natural materials” are any product or physical matter that comes from plants, animals or other materials existing in nature have not been substantially altered chemically or physically to alter their properties.

The terms “substantially dry” or “dried” describe a material that has a moisture content that is at most 10% by weight of the total weight of the sample, as measured using wet to dry weight or hygroscopic analysis. Useful examples of “substantially dry” or “dried” include less than less 5% wt/wt of moisture content, preferably less than 4% wt/wt of moisture content, more preferably less than 3% wt/wt of moisture content, even more preferably less than 2% wt/wt of moisture content, further preferably less than 1% wt/wt or even less than 0.5% wt/wt of moisture content, and most preferably less than 0.1% wt/wt or even 0% wt/wt of moisture content in a given material sample.

Accordingly, an approach was chosen that involved the possibility of utilizing common, and largely natural, food materials for fabrication of electronic components and electronic devices. It was believed that materials derived from natural foods may serve as the predominant elements in the fabrication of electronic components and electronic devices, and that any gaps in properties not provided by these materials may be filled in with edible processed food materials, food components, and, on a limited basis, non-toxic levels of traditional electronic materials to create full systems. Best candidate natural, processed, and adduct food materials were then selected to create a “preferred food kit” for electronic components and electronic device fabrication.

Therefore, to identify specifications of materials appropriate for fabrication of elements of edible/implantable electrical systems, some reference materials were initially selected. Food-related materials were obtained from public commercial markets, chain supermarkets or specialty food stores. Additive chemicals and agents were obtained from standard scientific suppliers and or hardware stores.

### Example 1: Dielectrics and Conducting Materials

Typical electronic circuits require dielectrics and conductors (identifies by the ranges of their electrical conductivities). A dielectric is known to be either an insulating material (insulator) or a very poor conductor of electric current. (When dielectrics are placed in an electric field, practically no current flows in them because, unlike metals, they have no loosely bound, or free, electrons that may drift through the material. Instead, electric polarization occurs.) Here, dielectric materials and insulating materials (which terms are used substantially interchangeably herein) were considered to be those with a conductivity σ lower than 10⁻⁸ S/m; while materials with a conductivity larger than 10⁶ S/m were defined to be electrically conducting or conductors. These reference σ values were used to establish the thresholds needed for systematizing the electronic components and electronic devices using food-based materials.

According to the scope of the invention, the combination of conductors and insulators is used for fabrication of active and passive microelectronic components of the edible and/or implantable electrical systems, while insulators are used to form coating layers and encapsulating layers (protecting at least a portion of a given embodiment of the electrical system from being very quickly dissolved inside the biological tissue) as well as the dielectric materials in a capacitor components of the electrical systems, (with capacitance in the typical range of 1 pF to 100 nF). The conductors appear in wires/interconnects, electrodes, and other components. A mix of dielectric and conducting food-based materials can be used to build resistors with a wide range of resistance from about 10Ω to about 20 MΩ. These reference values establish the specifications needed for components and devices fabrications using food materials.

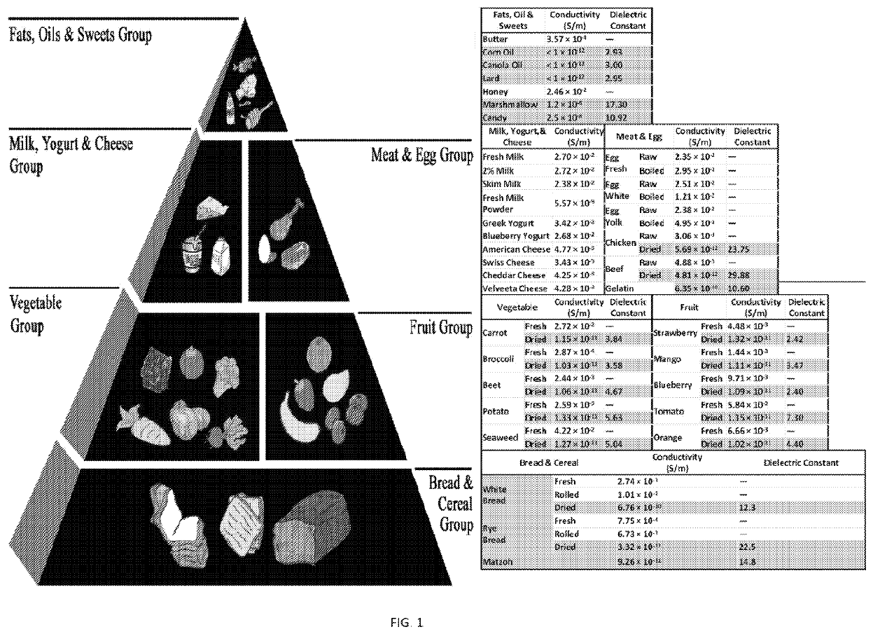

Specific natural, unprocessed foods, were selected, organized according to recognized defined food groups (e.g., fats, meat, vegetables, bread, etc.), as candidate materials for analysis as to electrical properties and subsequent component or device fabrication. Conductivity probes and semiconductor parameter analyzer were used to perform the characterizations. As illustrated by the shaded portion of the table of FIG. 1, the corresponding oils and dried foods (including meat, vegetables, gelatin, fruits, and bread) that are shaded can achieve required conductivities as insulators/dielectric materials. The natural foods are shown to be able to provide good insulators/dielectric materials, but might not perform sufficiently well as for electrical components. Here gelatin was cataloged into meat as it is derived from collagen in animal raw materials. The dried foods were made using a typical dryer. The reason for being good insulators is that natural foods contain covalent materials and do not contain mobile electrons to conduct electric current. On the contrary, foods that contain salts (e.g., butter) or water (e.g., fresh meat and vegetables) are relatively conductive because of the presents of free ions to conduct electric current.

A more comprehensive list of electrical conductivity and dielectric constants of commonly accessible food materials are summarized in Tables 1, 3, 6 below.

In order to fill the gap in the conductivity spectrum, food stuffs and non-toxic levels of electronic materials were similarly identified and analyzed. To this end, Table 2 illustrates parameters of some of conventional materials that are used in microelectronic fabrication.

### Example 2: Piezoelectrics

In some forms, an electronic component can include a piezoelectric material. Piezoelectric materials are known to generate voltage upon being exposed to mechanical stress. As such, piezoelectric materials can be used with other materials for sensing applications or for power generation, or in applications including pressure sensors, microphones, and speakers. Some natural and edible materials, such as bones and tendons, and cellulose (contained in many vegetables) display piezoelectric effects. The piezoelectric mechanism displayed by vegetable materials stems from the ability of oriented cellulose crystallites in vegetables exhibit shear piezoelectricity due to the internal rotation of polar atomic groups associated with asymmetric carbon atoms.

Accordingly, for the purposes of the implementations of the present idea, in some forms a piezoelectric material includes a composite that contains gelatin and a cellulose-rich vegetable. In some forms, a piezoelectric material is formed from cellulose, cellulose-rich vegetables, or a combination thereof. Examples of cellulose-rich vegetables include, but are not limited to, broccoli, cauliflower, cabbage, Brussels sprouts, spinach, lettuce, etc. In some forms, a plasticizer can be included into the composite in order to enhance the mechanical flexibility. Preferably, the plasticizer is edible. Examples of edible plasticizers include, but are not limited to, glycerol, propylene glycol, sorbitol, etc.

### Example 3: Binding Materials

In some forms, binding materials or binders can be used to increase the adhesion between elements of the implementations of electrical systems. Preferably, the binder is an edible binder. Suitable edible binders include, but are not limited to, egg whites and carboxymethyl cellulose. The presence of hydrogen bonds and ionic interactions with proteins gives rise to high adhesive strengths and allows egg whites as good adhesive materials. For example, binders from an embodiment of the set of materials can be used to achieve good adhesion between a substrate and a material being deposited on the substrate.

### Example 4: Encapsulating Materials

Embodiments of a set of materials for intended use, according to the idea of the invention, may include an encapsulant (configured as a coating layer, for example, covering an edible/implantable component of the electrical system to slow down the dissolution or absorption of such component inside the living biological tissue). Non-limiting examples of materials identified for use an encapsulating (coating) materials are shown in Table 4. In some cases, the insulating materials discussed above can also be used as encapsulants.

### Example 5: Additional Materials

Other materials can be included in the electrical systems. These include, but are not limited to, food dyes (e.g. FD &C Blue 1 brilliant, Blue FCF, FD & C Yellow 5, tartrazine, FD & C Red 3 erythrosine, and FD & C Red 40), and some semi-conductor materials such as phosphorus-doped silicon, boron-doped silicon, germanium, silicon-germanium alloy. It is to be understood that these additional materials should not exceed their safe dose when juxtaposed with the living tissue. For example, germanium is highly toxic and can cause irreversible detrimental effects at 150 ppm.

TABLE 1
A list of commonly accessible food materials and
itemized electrical characteristics.

| | | Conductivity | Dielectric |
|---|---|---|---|
| Dielectric materials | | (S/m) | Constant |
| Fats, Oil & | Gummy Bears | 5.78 × 10⁻⁵ | — |
| Sweets | Chewing Gum | 4.79 × 10⁻⁸ | 23.10 |
| | Sugar | 2.58 × 10⁻⁹ | 9.53 |
| | Glucose | 1.01 × 10⁻⁹ | 4.78 |
| | Dextrose | 1.40 × 10⁻⁹ | 5.26 |
| | Molasses | 7.14 × 10⁻⁴ | — |
| | Karo Syrup | 1.25 × 10⁻³ | — |
| Meat & Egg | Chicken (Cooked) | 9.43 × 10⁻⁴ | — |
| | Beef (Cooked) | 4.74 × 10⁻⁴ | — |
| Vegetable | Guar Gum | 5.2 × 10⁻⁹ | 9.99 |
| | Xantban Gum | 8.43 × 10⁻¹⁰ | 16.30 |
| | Kale (Fresh) | 6.42 × 10⁻⁶ | — |
| | Kale (Dry) | 2.25 × 10⁻¹¹ | 6.58 |
| | Cauliflower(Fresh) | 3.11 × 10 [symbol] | — |
| | Cauliflower (Dry) | 2.04 × 10⁻¹¹ | 4.97 |
| | Cucumber (Fresh) | 8.83 × 10⁻⁴ | — |
| | Cucumber (Dry) | 0.86 × 10⁻¹¹ | 5.37 |
| Fruit | Banana (Fresh) | 4.79 × 10⁻³ | — |
| | Banana (Dry) | 1.12 × 10⁻¹¹ | 6.00 |
| | Pineapple (Fresh) | 1.75 × 10⁻³ | — |
| | Pineapple (Dry) | 1.38 × 10⁻¹¹ | 4.56 |
| | Avocado | 2.48 × 10⁻³ | — |
| Bread & | Flour | 5.67 × 10⁻¹⁰ | 6.32 |
| Cereal | Corn Starch | 2.93 × 10⁻¹⁰ | 7.95 |
| Processed | Carbonized cotton | 35.07 | |
| food | Carbonized cotton candy | 22.46 | |
| | Carbonized silk | 28.29 | |

[symbol]

indicates data missing or illegible when filed

TABLE 2
Electrically-conducting materials and their safe dose
for use with a living tissue.

| | Sub- | Mobility | Conductivity | | |
|---|---|---|---|---|---|
| Category | categories | (cm²/(V.s) | (S/m) | Safe dose | |
| Carbon | Carbon | 100,000 | | | |
| | nanotubes | | | | |
| | Graphene | 10,000 | 1 × 10⁸ | | |
| | Active | | 21.18 | 100 | g/day |
| | charcoal | | | | |
| Edible | Solid Silver^{a} | | 6.3 × 10⁷ | 1.1 | mg/kg |
| Metal/ | Gold^{a} | | 4.1 × 10⁷ | 1.1 | mg/kg |
| Non- | Tungsten^{b} | | 1.79 × 10⁷ | 62.5 | mg/kg/day |
| metal | Magnesium^{c} | | 2.30 × 10⁷ | 11 | mg/day |
| | Iron^{d} | | 1 × 10⁷ | 45 | mg/day |
| | Chromium^{c} | | .90 × 10⁶ | 25 | ug/day |
| | Zinc^{c} | | 1.69 × 10⁷ | 40 | mg/day |
| | Boron^{c} | | 1.00 × 10⁴ | 20 | mg/day |
| | Calcium^{e} | | 2.98 × 10⁷ | 2500 | mg/day |
| | Molybdenum^{c} | | 2.00 × 10⁷ | 2 | mg/day |
| | Copper^{c} | | 5.96 × 10⁷ | 900 | ug/day |
| | Phosphorous^{f} | | 1.00 × 10⁷ | 700 | mg/day |
| | Cobalt^{g} | | 1.70 × 10⁷ | 10 | mg/kg |
| | Nickle^{e} | | 1.43 × 10⁷ | 1.0 | mg/day |
| | Platinum^{e} | | 2.38 × 10⁶ | | |
| | Vanadium^{e} | | 5.00 × 10⁶ | 1.8 | mg/day |
| | Sodium^{e} | | 2.10 × 10⁷ | 2.3 | g/day |

^{a}Fulati, et al., Sensors, 2009,9, 8911-8923;
^{b}McInturf, et al., Toxicol. Appl. Pharmacol., 2011, 254, 133-137 (2011);
^{c}Table and Table, Dietary reference intakes for vitamin A, vitamin K, arsenic, boron, chromium, copper, iodine, iron, manganese, molybdenum, nickel, silicon, vanadium, and zinc. (2001);
^{d}Erdman Jr, I. A. MacDonald, S. H. Zeisel, Present knowledge in nutrition. (John Wiley & Sons, 2012);
^{e}Del Valle, et al., Dietary reference intakes for calcium and vitamin D. (National Academies Press, 2011);
^{f}Jain and Elsayed, J Nephrol., 2013, 26, 856-864;
^{g}Wildman & Medeiros, Advanced human nutrition. (CRC press, 1999).

TABLE 3
Dielectric materials

| Physical | Dielectric | Sub- | |
|---|---|---|---|
| form | material | categories | Safe dose |
| Dry | Sugar | Glucose | <10% daily energy 90 g/d |
| | | Dextrose | |
| | | Fructose | |
| | | Caddy candy | |
| | Protein | Egg yolk | Female: 1-3 y: 13 g. 4-8 y: 19 g. |
| | | Egg white | 9-13 y: 34 g. 14+ y: 46 g Male: |
| | | Gelatin | 1-3 y: 13 g. 4-8 y: 19 g. 9-13 y: |
| | | | 34 g. 14-18 y: 52 g. 18+ y: 56 g |
| | Cheese | Sharp cheddar | 35.3-70 g/d Total fat(key |
| | | Mozzarella | component): 15%-30%. Saturated |
| | | Milk mutiara | fatty acids: <10%. Polyunsaturated |
| | | cheese | fatty acids 6-10%. Trans fatty acids |
| | | Other cheeses | < 1% Sodium(key component) |
| | | | 2.3 g/day Protein: Female: 1-3 y: |
| | | | 13 g. 4-8 y: 19 g. 9-13 y: 34 g. |
| | | | 14+ y: 46 g Male: 1-3 y: 13 g. |
| | | | 4-8 y: 19 g. 9-13 y: 34 g. 14-18 y: |
| | | | 52 g. 18+ y: 56 g cholesterol (key |
| | | | component): <300 mg/day |
| | Gum | Gummy bears | Carbohydrate(key component): |
| | | Chewing gum | 281-384 g/d 55-75% |
| | | bazooka | |
| | | Guar gum | |
| | | Xanthan gum | |
| | Oil | Butter | Fat, Potassium, Sodium, Protein, |
| | | | Carbohydrate |
| | Clif Bar | | Fat, Cholesterol, Potassium, |
| | | | Sodium, Protein, Carbohydrate |
| | Shot Bloks | | sodium, potassium, carbohydrate |
| | Flower | | |
| | Si₃N₄ | | |
| | SiO₂ | | |
| Powder | Flour | | Carbohydrate(key component): |
| | | | 281-384 g/d 55-75% |
| | Corn starch | | Carbohydrate(key component): |
| | | | 281-384 g/d 55-75% |
| | Protein | Collagen | Female: 1-3 y: 13 g. 4-8 y: 19 g. |
| | | Zein | 9-13 y: 34 g. 14+ y: 46 g Male: |
| | | BSA | 1-3 y: 13 g. 4-8 y: 19 g. 9-13 y: |
| | | Soy protein | 34 g. 14-18 y: 52 g. 18+ y: 56 g |
| | | Whey | |
| Solution | Oil | Corn oil | 35.3-70 g/d Total fat(key |
| | | Other oil | component): 15%-30%. Saturated |
| | | | fatty acids: <10%. Polyunsaturated |
| | | | fatty acids 6-10%. Trans fatty acids |
| | | | < 1% |
| | Honey | | key component: Sugar: <10% daily |
| | | | energy 90 g/d Carbohydrate³: 281- |
| | | | 384 g/d 55-75% |
| | Sugar | Karo syrup | <10% daily energy 90 g/d |

TABLE 4
Non-limiting list of materials identified for use as encapsulating materials

| Category | | Safe does |
|---|---|---|
| | Cheese | 35.3-70 g/d Total fat (key |
| | | component): 15%-30%. Saturated |
| | | fatty acids: <10%. Polyunsaturated |
| | | fatty acids 6-10%. Trans fatty acids |
| | | < 1% Sodium(key component) 2.3 g/day |
| | | Protein: Female: 1-3 y: 13 g. 4-8 y: |
| | | 19 g. 9-13 y: 34 g. 14+ y: 46 g Male: |
| | | 1-3 y: 13 g. 4-8 y: 19 g. 9-13 y: 34 g. |
| | | 14-18 y: 52 g. 18+ y: 56 g cholesterol |
| | | (key component): <300 mg/day |
| | EUDRAGIT ® | |
| | Aspirin or enteric | |
| | coating | |
| | Gum | Carbohydrate(key component): 281- |
| | | 384 g/d 55-75% |
| | Meat | |
| | Fat | 35.3-70 g/d Total fat: 15%-30%. |
| | | Saturated fatty acids: <10%. |
| | | Polyunsaturated fatty acids 6-10%. |
| | | Trans fatty acids < 1% |
| Protein + Fat | | |
| | Polymer | |
| Complex | Mixture of sugar | |
| encapsulate | and materials such | |
| | as protein | |
| | Other mixtures | |

### Characterization of Various Materials for Identification of Materials Suitable for Formation of Material Sets for Use in Fabrication of an Embodiment of an Electrical System.

### Example 6: Characterization of Conductive/Dielectric Materials

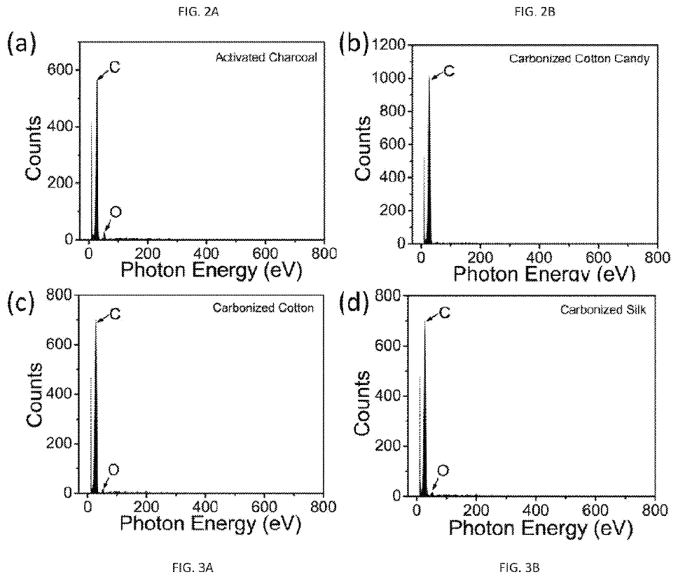

In addition to edible non-toxic metals, carbon derived from processed foods (in practice—substantially activated charcoal), carbonized sugar (cotton candy), cellulous (cotton), and protein (silk) were selected and tested. Annealing process was used for carbonization of a given material. As illustrated in FIGS. 2A, 2B, 3A, 3B, the energy dispersive X-ray spectrometry (EDX) results show that these materials contain carbon.

The surface morphology and element content of activated charcoal, carbonized cotton candy, carbonized cotton, carbonized silk, broccoli powder, piezoelectric film, inductor and conducting material were determined using scanning electron microscopy (Hitachi S4700 FESEM).

The results, including different microscale morphologies of carbonized cotton candy (FIG. 4), cotton (FIG. 5), and silk (FIG. 6), are attributed to different electrical properties of these materials. Specifically, the fiber-liked carbonized cotton tends to form a continuous path to conduct electrons while the flake-liked carbonized cotton candy and silk have to aggregate to form the similar conductive path.

Some materials were coated with 200 nm thick layer of gold in a vacuum chamber prior to taking SEM, EDX images.

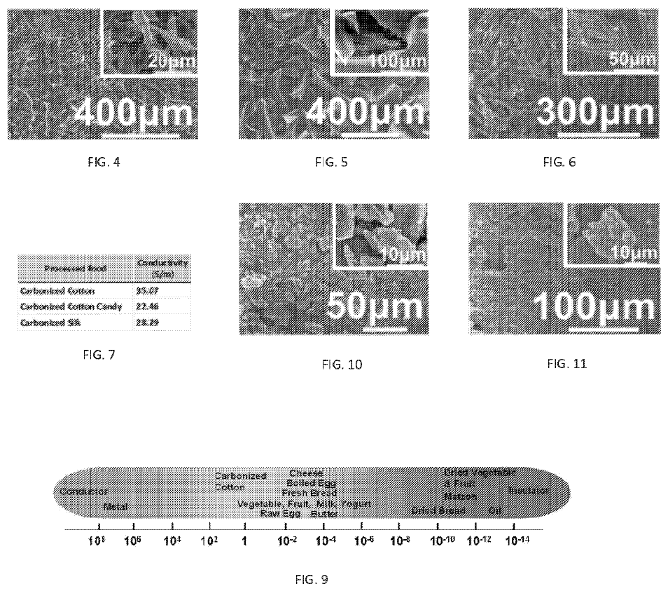

The electronic conductivity results summarized in FIG. 7 show that these processed food materials and non-toxic metals (see, also, Table 2) can serve fir use as intended, as the conductive materials for fabrication of embodiments of electrical systems. It is apparent from FIGS. 4-7 and Table 2 that edible food materials can cover a wide range of electrical conductivity, summarized in FIG. 9. To build a conductive wire/interconnect, edible metals are used while dried vegetables mixed with bread/flour and oil are used for insulators. The mixed carbonized cotton candy and flour can be used to build resistors.

### Example 7

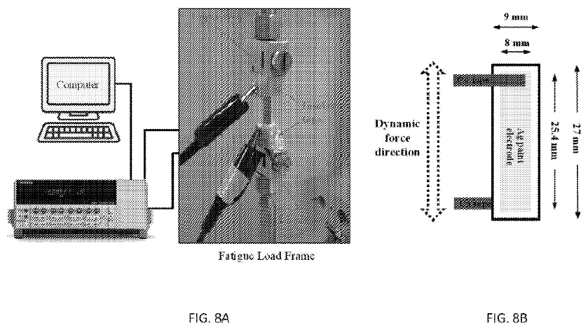

Characterization of piezoelectric materials appropriate for use as intended was carried out with broccoli powder containing power conglomerations with radius smaller than 50 μm (mixed with gelatin to form a piezoelectric composite). FIG. 10 illustrates an SEM image of the broccoli powder.

In some embodiments, a piezoelectric material from the set of materials can be formatted as a thin film (or shaped differently). Broccoli powders (Holistic Herbal Solutions, LLC) were sieved through a sieve with mesh size 90 μm and then was uniformly mixed with gelatin solution through magnetic stirring, followed by casting at 24° C. 2 g of gelatin was used for every 1 g of broccoli powders. The stiffness of the thin film was adjusted by adding an edible plasticizer, such as glycerol. The piezoelectric coupling coefficients of the edible piezoelectric thin film were characterized by using an electric shaker, accelerometer and signal analyzer. The coupling coefficients were measured to be d₃₃=4.3 pC/N, and d₃₁=0.31 pC/N. These empirically-defined values were comparable to 5 pC/N of ZnO, which is a well-known piezoelectric material. FIG. 11 shows a cross-sectional view of the edible piezoelectric thin film made with broccoli powder.

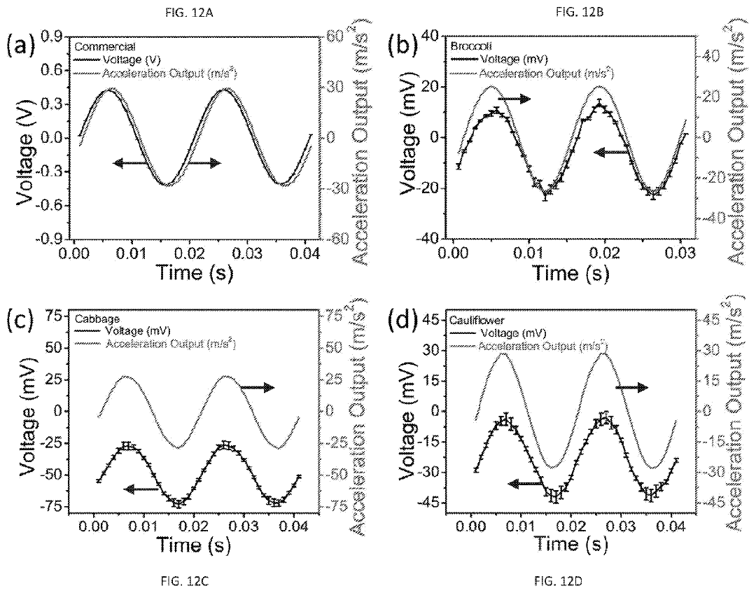

In addition to broccoli that is rich in cellulose, other cellulose-rich foods, including Brussels sprouts, cauliflower, and cabbage were also mixed with gelation to form piezoelectric composites using the approach described herein. The same approach described above, was used in the characterization of the piezoelectric performances of these materials. The results are shown in FIGS. 12A, 12B, 12C, and 12D, and are shown in Table 7.

The materials all exhibited appreciable piezoelectric effects. Under the same weight ratio between cellulose-containing vegetables and gelatin (1 g cellulose-containing vegetable to 2 g gelatin), broccoli has the strongest piezoelectric effects since it is the most cellulose-rich vegetable. It should be noted that gelatin also has detectable piezoelectric effects since it is derived from collagen from animal raw materials. However, its d₃₃ coefficient is about 30 times less than that of broccoli. Therefore, the observed piezoelectric effects are primarily defined by the material(s) of the vegetables.

A skilled artisan will readily appreciate, therefore, that the studies of food materials with respect to their electrical properties opens opportunities to build a toolkit of electrical components necessary for formation of embodiments of the edible/implantable electrical systems.

Examples of Electrical Systems and Constituent Components and/or Devices.

The electrical systems described herein encompass electronic components and electronic devices. The electronic components are fabricated using one or more of the food materials, processed food materials, food-like materials, foodstuffs, and natural materials, or a combination thereof. Preferably, the electronic components include at least one or more food materials.

One or more of the electronic components, operably cooperated with one antother, may be used to fabricate the electronic devices. Embodiments of the electrical systems are edible and/or ingestible and/or biocompatible and/or biodegradable and/or bioresorbable and/or implantable

Embodiments of the discussed below electronic components degrade, with time, inside the living tissue (such as the GI tract, if generally introduced into a subject). The nature of degradation can includehydrolytic, enzymatic, thermal processes or a combination thereof. The rate of degradation can be varied and/or predetermined by the choice of materials, the volume, mass, density of the materials used to form the constituent electronic components, the presence of encapsulating layer(s), or a combination of several such means.

Representative constituent components/devices of embodiments of electrical systems include capacitors, inductors, antennas, interconnects (e.g. a conducting material wire), insulators, resistors (e.g. a potentiometer), transistors, diodes (e.g. light-emitting diodes), conducting materials/interconnects, printed circuit boards, electrodes, and piezoelectric elements.

### Example 8: Capacitors

According to one embodiment, a capacitor was fabricated using pieces of conductive materials arranged in parallel, and separated by one or more pieces of dielectric materials. Preferably, the conducting materials are formed into thin plates or patterns deposited on opposite sides of the dielectric materials. The material may be applied via manual application, painting on, spraying on, brushing, souring with evaporation, solvent evaporation with an organic (e.g. alcohol), spin casting the deposited conducting material and dielectric materials function as current or charge collectors and dielectric or separator, respectively. When the conducting materials are deposited as thin plates, these plates preferably form a layer over the entire face of the dielectric material on which they are deposited. Preferably, the used conducting materials are inert, non-toxic, i.e., these materials do not take part in a chemical reaction when introduced into a subject. An edible, non-toxic metal such as gold (for example, with a purity of 23 karat) is an example of ac conducting material used for fabrication of an edible capacitor. The thickness of the metallic conducting material layer on each surface of the edible dielectric material can be between 50 nm and 500 nm, inclusive, between 100 nm and 400 nm, or between 150 nm and 300 nm, inclusive. In some implementations, the thickness of the metal conducting material layer is about 200 nm. (A list of conducting materials that can be used to make the plates of the capacitor are shown in Table 2.)

Plethora of dielectric materials that can be used to make the capacitor are shown in Table 3. In some forms the dielectric material contains an edible plasticizer, such as glycerol, or propylene glycol, to enhance the mechanical flexibility of the material. In some forms, a high-k material can also be added to dielectric to increase the dielectric constant of the resulting composite dielectric material. In some implementations, the dielectric material is molded as a film, which can have a thickness between 10 μm and 500 μm, inclusive, preferably between 80 μm and 140 μm, inclusive. Preferably, the dielectric material contains gelatin.

The capacitors described above can have a capacitance between 1 pF and 200 nF, inclusive, preferably between the typical range of 1 pF and 100 nF, inclusive. It is understood that the total surface area of the deposited conducting material (plate or patterned), the thickness of the dielectric between the deposited conducting materials, or both, can be varied as needed to alter the capacitance of the capacitors.

In one specific implementation, the resulting capacitor additionally an electrolyte material that serves as a charge carrier when a voltage is applied across the capacitor

In one representative case, a gelatin film was used as the dielectric layer. Here, 2 g of gelatin was weighed and sprinkled over 20 g of distilled water. The mixture was keptfor 10 min, in order for the gelatin to fully swell, after which a gelatin solution was prepared by dissolving the gelatin in approximately 100 ml distilled water with constant magnetic stirring for 30 min. The temperature of the gelatin solution was maintained at 60° C. during the mixing and stirring. Subsequently, 1.2 g of glycerol were weighed and added to gelatin solution and stirred for 10 min. The glycerol served as an edible plasticizer. Gelatin films were produced by solution casting on acrylic glass plate at 50° C. for 12 hrs. The typical thickness of the gelatin film varies from 80 μm to 140 μm. The pattern of the metal trace was defined by a shadow mask. Two shadow masks having the same pattern were carefully aligned and attached to both sides of the gelatin film. A 200-nm thick layer of gold was deposited each side of the gelatin film using gold sputter. The shadow masks were removed after the deposition to form the resulting capacitor. Here, the deposited gold and gelatin film served as the current or charge collector and dielectric or separator, respectively.

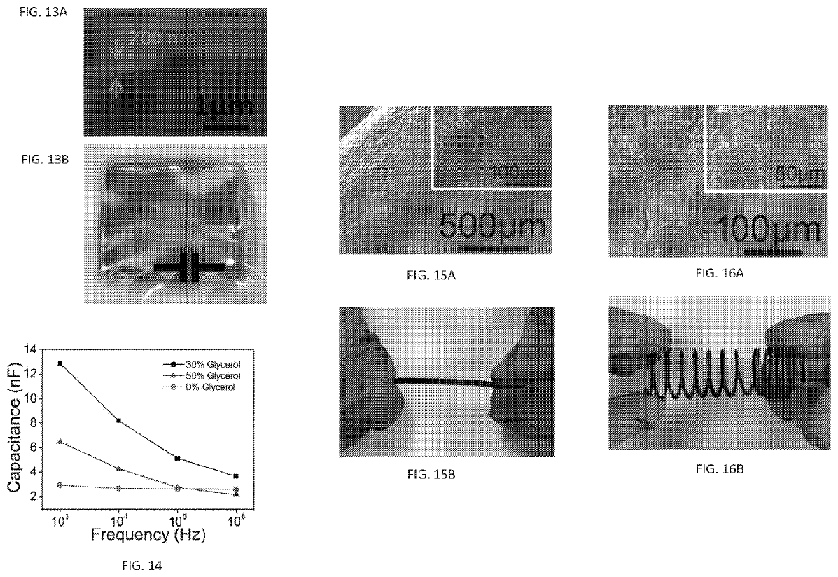

FIGS. 13A, 13B contain two images, the first of which shows a cross-section through an embodiment of the capacitor and the second of which presents the outside view of the capacitor made if thin gelatin sheets as dielectric layers coated with edible Au as the electrodes. Measurements of the capacitance were carried out on a probe station with precision LCR meter (Hewlett-Packard 4061A semiconducting material/component test system). The capacitance of capacitors built with different compositions of an edible plasticizer, glycerol, are shown in FIG. 14.

### Examples 9 and 10: Resistors and Inductors

Mixed insulator-conducting material systems including materials from the set(s0 described above can be used to devise resistors. The resistors can have a resistance ranging between 0.1Ω and 25 MΩ, inclusive, preferably between 10Ω and 20 MΩ, inclusive. In some embodiments, the resistor was made using an insulating material (such as sweet potato starch) and a conducting material (such as carbonized cotton candy) through an extrusion process using a syringe.

In one embodiment, the resistors were formatted as straight wires, i.e., conductive “noodles.” One or more layers of the conducting material can be added on the outside of the resistors while they are still wet, during the fabrication process, to increase the conductivity.

However, during the extrusion step the building material can be wound around an object of a chosen shape. When the objects (pre-form) is cylindrical, an inductor results. In some forms, the length of the inductor can be between 2 mm and 30 mm, preferably between 5 mm and 20 mm. In some embodiments, the diameter of the inductor loop is between 2 mm and 40 mm, inclusive, preferably between 5 mm and 30 mm. In some forms, the inductance of the inductor can be between 0.5 μH and 10⁴ μH, inclusive. It is understood, that the length, diameter, or both, of the inductor can be adjusted to achieve desired inductance values.

In one case, to produce a resistor, 1.5 g of sweet potato starch, 0.45 g of active charcoal, and 0.5 g of carbonized cotton candy were weighed and mixed together in separate ceramic containers. The mixture was ground for 10 min to uniformly disperse the sweet potato starch, active charcoal, and carbonized cotton candy. The container was then put on a hotplate (at 200° C.) and heated for 5 min, followed by adding 2 g of distilled water, and heating for another 5 min under constant stirring. The heated mixture formed a low-viscosity (slightly runny) dough. The mixture was then transferred into a syringe. Syringes having different orifice sizes can be used. The size of the orifice defines the diameter of the edible resistors. The typical used orifice diameters were 2 mm, with a range between 0.1 and 2 mm (i.e., largest orifice corresponded to 18 gauge). The mixture was pushed out at a rate of 0.7 mL/min into boiled distilled water for 3 min and then immersed in the distilled water at room temperature to increase the tenacity/stiffness of the extruded mixture. Finally, the extruded mixture was delivered into an oven and dried at 60° C. for 12 hrs to form edible resistors. The edible resistors are, essentially conductive and edible noodles.

Edible resistors can also be formed from carbonized cotton and carbonized silk following the procedure described above, while replacing the carbonized cotton candy with these materials. Flour can also be used in place of sweet potato starch in this process.

The edible inductors were prepared following the procedure described above for making edible resistors. After the mixture was pushed into the boiled distilled water and immersed in the distilled water at room temperature, the sample was wound around a cylindrical object such as—a simple metal or glass rod or cylindrical tube having a diameter of between about 2 and 15 mm, with lengths between 40 cm and 80 cm. The winding length and diameter of the cylindrical object influence the inductance of the edible inductors. The wound edible inductor was then dried in an oven at 60° C. for 12 h, and subsequently taken off from the cylindrical object after the drying process. The edible inductor was produced substantially dry.

FIGS. 15A, 15B and FIGS. 16A, 16B illustrate SEM and optical images of resistors and inductors, respectively, made of sweet potato starch and carbonized cotton candy through the described extrusion process. As shown in the SEM images of FIGS. 15A, 16A, carbonized cotton forms a substantially continuous electrical patch along the body of the “noodles”.

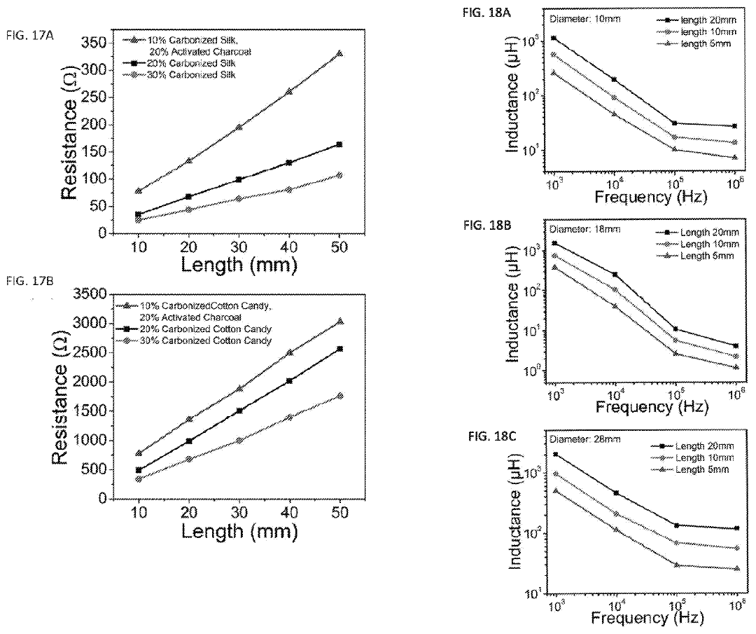

FIGS. 17A, 17B contain line plots of resistance test results formed from different composite materials as compared to that formed from active charcoal, as a function of length. Carbonized silk (FIG. 17A) and carbonized cotton candy (FIG. 17B) were used as a substitute for carbonized cotton. The content of carbonized silk and carbonized cotton candy was 10%, 20%, or 30%.

FIGS. 18A, 18B, 18C are line graphs showing the operational performance of inductors with different diameters and lengths under frequencies between 10³ Hz and 10⁶ Hz, inclusive. The diameterd of the coil str 10 mm (FIG. 18A), 18 mm (FIG. 18B), and 28 mm (FIG. 17C). The inductors produced using food-based materials, display properties that are similar to those of non-food-based materials: for a given frequency scan, inductance is directly proportional to the length and diameter of the inductor.

### Example 11: Wires/Interconnects

In certain embodiments, the detailed fabrication approaches are substantially the same as described above in reference to resistors. Briefly, one of edible wire was made of rice paper as the substrate material and sputtered Au as the functional part. See FIG. 19A. The edible wire and interconnects discussed in the examples above were formed as a substrate with a layer of conductive trace. The substrate was made of rice paper or any other food-based material that has resistivity larger than 1×1010Ω·m. The dimension of rice substrate was 3 mm×(20-50 mm)×0.2 mm (thickness). A shadow mask with the pattern of the interconnect/wire was attached to the rice paper using egg white as the adhesive layer. Embodiments then were dried in oven at 70° C. for 8 hrs to form substrates.

The substrate was then placed in a vacuum chamber of gold sputtering machine, where gold was deposited on the substrate through the shadow mask, with thickness of 100 nm. Rice paper is very thin and thus very flexible so that it is used as the substrate in wires/interconnects. The thickness of the Au layer was on the order of 100 nm, as shown in an SEM cross-sectional image of FIG. 19B, and the resistance of such wire as a function of length is presented in FIG. 19C.

In a related embodiment, wires were prepared by coating rice paper as a substrate with gold in a gold sputterer for 1 min, 5 mins, and 10 mins, and the resistance of these wires was determined as a function of their length, FIG. 31. As shown in FIG. 31, wires produced by coating the substrate with gold for 1 min had the highest resistance (three-fold) over the range of lengths tested. Meanwhile, substrates coated with gold for 5 mins and 10 mins had similar resistance over the lengths tested. This shows that less gold can be used (5-min coating) to achieve the same electric properties than a higher quantity (10-min coating).

### Example 13: Printed Circuit Board/Bread Board

Edible printable circuit boards (PCBs) facsimiles served as a substrate on which other components of the edible electronic device were mounted. Preferably, the PCB is made edible. In some forms, an edible PCB substrate can be formed from materials such as powdered sugar, xanthan gum, and egg white. Prior to mounting an electrical system on the edible PBC, a binder can be applied to the surface of the PCB to ensure good adhesion between the edible PCB and the electrical system.

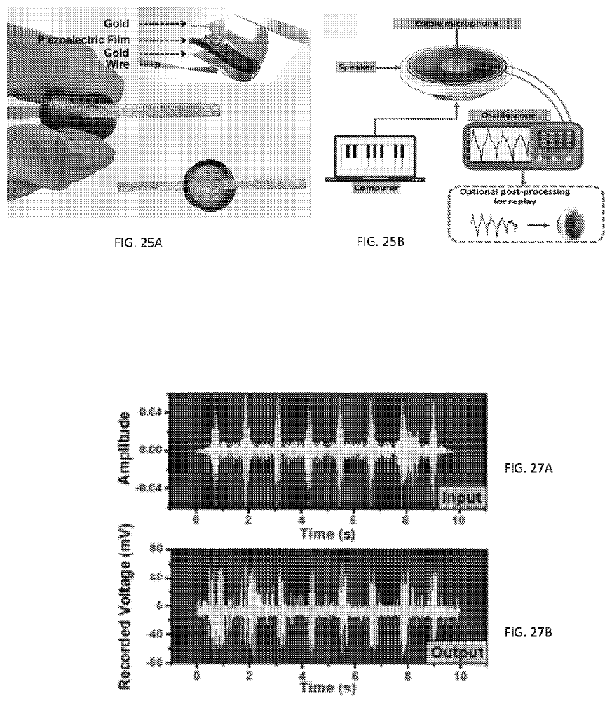

Each edible PCB substrate was made using powdered sugar, xanthan gum, and egg white as follows: 60 g of powdered sugar, 0.5 g of xanthan gum, and 12 g of egg white were weighed and mixed together in a glass bowl using a hand mixer. They were mixed until a sticky paste was formed, and most of the powdered sugar had been incorporated into the sticky paste. 20 g of additional powdered sugar and the sticky paste were poured on a lab workbench, followed by kneading until a smooth and non-sticky sugar paste dough was formed. The non-sticky sugar paste dough was divided into eight pieces. Each of the pieces was rolled out and cut into a 7 cm×7 cm×0.2 cm piece or substrate (i.e., edible PCB substrate). Each cut piece was then dried at room temperature for 12 hrs. Before placing a shadow mask on an edible PCB substrate, a uniform egg white layer was coated on the surface of the edible PCB substrate. The egg white served as a surface adhesive to ensure good attachment of the shadow mask to the edible PCB substrate. After drying the edible PCB substrate in an oven at 70° C. for 8 hrs, each PCB was placed in the vacuum chamber of a gold sputter, and gold was deposited on its surface against the shadow mask. FIG. 24A illustrates a top view of a PCB 2404 made of powdered sugar with deposited gold layers 2406.

According to embodiments of the invention, a bread board may be fabricated from a range of carbohydrate and protein constituents, these may be primarily formed or may be fabricated from partially processed foods. Examples include utilizing bread such as white bread which is rolled flat and compressed and then perforated to allow insertion of components. Alternatively, a food product such as matzoh, unleavened bread which is flat and cracker like with perforations may be utilized. Similar bland digestive or saltine crackers may be utilized. A perspective view of a bread board (cracker/saltine) 2410 with a conductor (carbonized resistor 2420 and inductor 2430—wound carbonized material) is shown in FIG. 24B.

### Example 14: Antenna

An antenna transmits and/or receives signals between electronic components that are physically and/or wirelessly connected. In some forms, the antenna transmits and/or receives signals from a source within and/or external to a subject. An antenna component can be fabricated using any of the conducting materials described above. In some forms, the antenna includes edible Au.

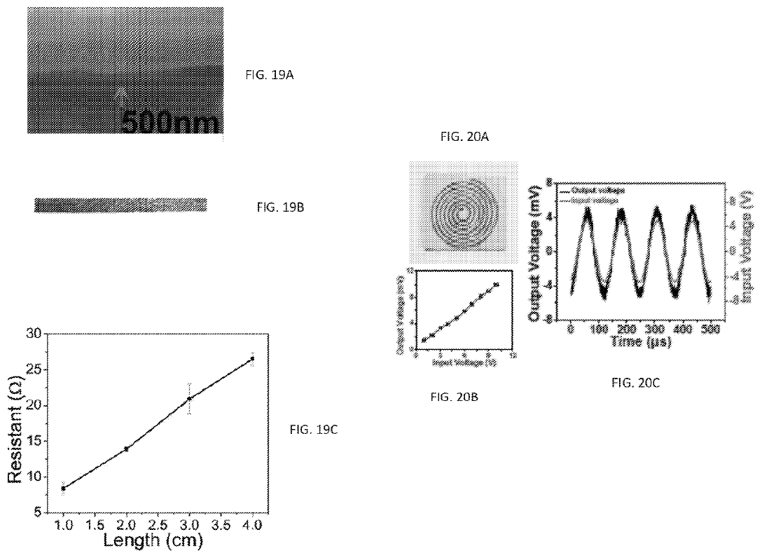

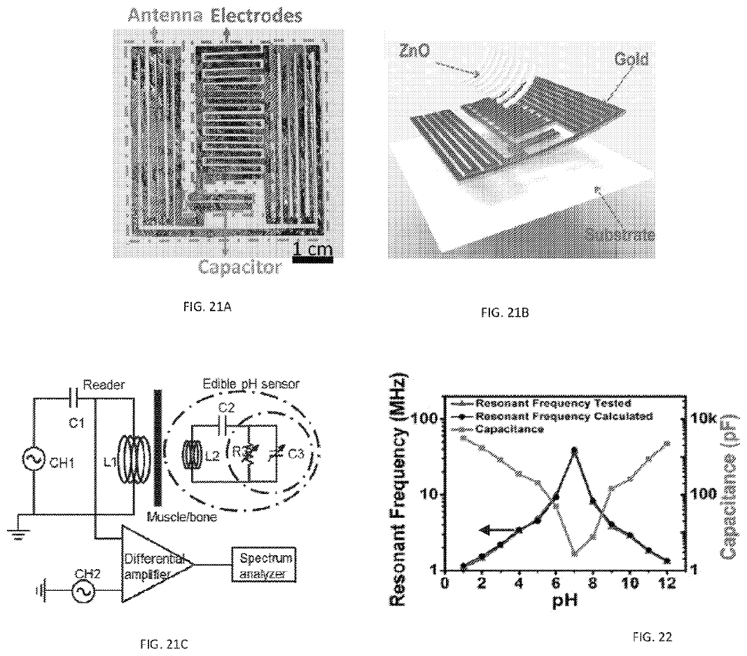

In practice, the shadow mask for the antenna was made using the same method described below in the section Preparation of shadow mask. Then the shadow mask was attached to the edible PCB substrate, followed by deposition of an approximately 200-nm thick layer of gold deposit using a gold sputter. Following deposition, the shadow mask was removed, and the antenna was formed on the edible PCB substrate. FIGS. 20A, 20B, 20C illustrate is a perspective view of an embodiment of an antenna and graphs of output voltage of the antenna as a function of input voltage and as a function of time, respectively.

To characterize an embodiment of the antrenna, a transmitter made of 50-turn copper coils (diameter—50 mm; height—60 mm) was connected to a signal generator, and an alternating current (AC) source was connected to the transmitter with peak-to-peak voltages between 1V and 10V, inclusive. The tested antenna was placed facing the cross section of the coil at a distance of 10 mm. The two ports of the tested antenna were connected to an oscilloscope, where signal waveforms and peak-to-peak values were recorded.

Additionally, several active systems, i.e., systems with specific functionalities, including a pH sensor, a radio frequency filter, and a piezoelectric microphone, were fabricated and tested.

### Example 15: A pH Monitoring Circuit (a pH Sensor)

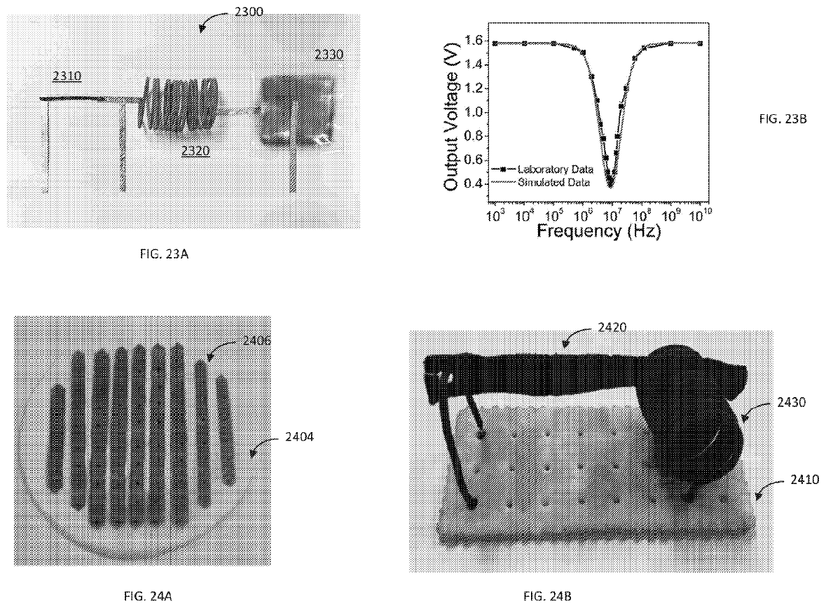

An embodiment of an edible pH sensor included thin layer of gold-zinc(II) oxide (Au—ZnO) as working electrodes, an antenna made of gold for wirelessly transmitting signals, and an edible capacitor, all disposed on an edible substrate (e.g., an edible matzoth substrate), FIGS. 21A, 21B. The reaction of ZnO with either acidic or basic solutions may be used to change the capacitance C between Au and ZnO electrodes, and thus the resonant frequency f of the pH sensor according to

f=1/(2π\sqrt{LC}, where L is the inductance of the antenna that does not depend on the pH value. To calibrate the edible pH sensor, the pH value of various solutions was measured by a standard lab pH meter; the capacitance of the Au—ZnO electrodes were characterized, and the resonant frequency of the pH sensor was detected using a commercially available circuit made of a reader, a differential amplifier, and a spectrum analyzer FIG. 21C. During the calibration process, the edible pH sensors were immersed in testing solutions with pH values ranging from 1 to 14.

The working mechanism was that for acidic solutions. The H⁺ residing at the ZnO surface can protonate or deprotonate: ZnO_{(S)}+H_{S}⁺↔Zn(OH)⁺, leading to a surface charge and a surface potential, thus it is pH-sensitive. For basic solutions, with increasing OH⁻ groups, hydroxyl complexes such as Zn(OH)₃⁻ will appear, ZnO_{(S)}+2H₂O↔Zn(OH)₃⁻+H_{S}⁻. FIG. 22 provides plots illustrating empirically determined the capacitance and resonance frequency values of the embodiment of the edible pH sensor of FIGS. 21A, 21B, 21C.

### Example 16: Radio Frequency Filter

In reference to FIG. 23A, an edible radio frequency (RF) filter 2300 containing i) a resistor 2310 made of carbonized cotton candy and sweet potato starch; ii) an inductor 2320 made of carbonized cotton and sweet potato starch, and an iii) edible capacitor 2330 was fabricated and its frequency-dependent characteristics were tested. The values of the resistance (R), inductance (L), and capacitance (C) were chosen for the frequency range of interest, and were measured and verified individually (R=20Ω, L=0.2 μH, C=1.7 nF). This series RLC filter's frequency response was simulated, and measured with conventional signal generator and oscilloscope. The corresponding plots are presented in FIG. 23B

### Example 17: Piezoelectric Microphone

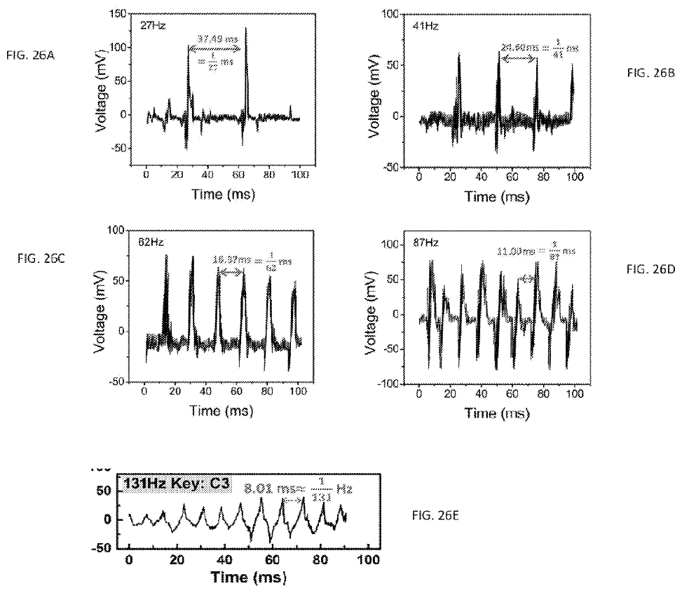

In reference to FIGS. 25A, 25B, 26A, 26B, 26C, 26D, 27A, and 27B, to determine whether the edible piezoelectric thin film could be used to convert mechanical vibration to appreciable voltage changes for potential biomedical applications, an edible piezoelectric microphone was built as follows: A 2 mm-thick edible piezoelectric thin film was formed using the gelatin/broccoli film described above under the section Piezoelectric composite thin films. The piezoelectric thin film was coated with 200 nm-thick Au electrodes on both sides. Conducting material wires, built by applying a 100-nm layer of gold to a rice paper, were attached to the Au electrodes.

To test the edible piezoelectric microphone, sounds with defined frequency generated from a computer (i.e., virtual piano keys) were played back via a loudspeaker, where the edible piezoelectric microphone was firmly attached to the loudspeaker's diaphragm to detect mechanical vibrations. The generated frequencies ranged between 27 Hz and 131 Hz (only several non-limiting examples of results are shown for simplicity). The embodiment of the edible microphone was connected to an oscilloscope to record and show the voltage waveform. The recorded analog voltage signals from the oscilloscope were further fed to a loudspeaker for optional playback. It is appreciated that low-frequency sound is particularly important in biomedical applications as it is within the range of abdominal associated with both normal and pathologic conditions. To determine whether the edible piezoelectric microphone could be used in a biomedical application, bowel sounds from a 70-year old man with abdominal pain were fed to the loudspeaker and recorded via the edible microphone. The recorded voltage waveform was compared to that of the original testing sound, with good correlation.

It is appreciated that the results of the overall study of food-related materials with respect to their electrical properties and demonstrations of fabrication of embodiments of edible/implantable electrical systems presented an opportunity for building a “toolkit” containing materials that can be used to fabricate edible electronic components and edible electronic devices, a non-limiting example of which as shown in Table 5.

TABLE 5
Materials in a toolkit for building electronic components

| Com- | Food Kit Materials | |
|---|---|---|
| ponent | Structural Function | Electrical Function |
| Wire | Rice Paper/Sugar | Gold leaf/Edible metal: gold |
| | powder/flour/rice | |
| Resistor | Sweet potato | Active Charcoal/carbonized |
| | powder/flour/Candy/Dried | cotton fiber/cotton candy/silk/ |
| | Fruit/Vegetable | Gold leaf |
| Inductor | Sweet potato | Active Charcoal/carbonized |
| | powder/flour/Candy/Dried | cotton fiber/cotton candy/silk/ |
| | Fruit/Vegetable | Gold leaf |
| Capacitor | Gelatin/Dried Fruit/Vegetable | Gold leaf/Edible metal: gold |
| Antenna | Sugar powder/flour/rice | Edible metal: gold/Gold leaf/ |
| | paper/Candy/Marshmallow, | Active Charcoal/carbonized |
| | egg white | cotton/carbonized silk |

TABLE 6
List of Foods with corresponding conductivities and dielectric constants

| Fats, Oil & | Conductivity | Dielectric |
|---|---|---|
| Sweets | (S/m) | Constant |
| Butter | 3.57 × 10⁻⁴ | — |
| Corn Oil | <1 × 10⁻¹² | 2.93 |
| Canola Oil | <1 × 10⁻¹² | 3.00 |
| Lard | <1 × 10⁻¹² | 2.95 |
| Honey | 2.46 × 10⁻² | — |
| Marshmallow | 1.2 × 10⁻⁸ | 17.30 |
| Candy | 2.5 × 10⁻⁸ | 10.92 |

| Milk, Yogurt, & | Conductivity | | | Conductivity | Dielectric |
|---|---|---|---|---|---|
| Cheese | (S/m) | Meat & Egg | | (S/m) | Constant |
| Fresh Milk | 2.70 × 10⁻² | Egg Fresh | Raw | 2.35 × 10⁻² | — |
| 2% Milk | 2.72 × 10⁻² | | Boiled | 2.95 × 10⁻³ | — |
| Skim Milk | 2.38 × 10⁻² | Egg White | Raw | 2.51 × 10⁻² | — |
| Fresh Milk Powder | 5.57 × 10⁻⁹ | | Boiled | 1.21 × 10⁻² | — |
| Greek Yogurt | 3.42 × 10⁻² | Egg Yolk | Raw | 2.38 × 10⁻² | — |
| Blueberry Yogurt | 2.68 × 10⁻² | | Boiled | 4.95 × 10⁻³ | — |
| American Cheese | 4.77 × 10⁻³ | Chicken | Raw | 3.06 × 10⁻³ | — |
| Swiss Cheese | 3.43 × 10⁻³ | | Dried | 5.69 × 10⁻¹² | 23.75 |
| Cheddar Cheese | 4.25 × 10⁻³ | Beef | Raw | 4.88 × 10⁻³ | — |
| Velveeta Cheese | 4.28 × 10⁻³ | | Dried | 4.81 × 10⁻¹² | 29.88 |
| | | Gelatin | | 6.35 × 10⁻¹⁰ | 10.60 |

| | | Conductivity | Dielectric | | | Conductivity | Dielectric |
|---|---|---|---|---|---|---|---|
| Vegetable | | (S/m) | Constant | Fruit | | (S/m) | Constant |
| Carrot | Fresh | 2.72 × 10⁻² | — | Strawberry | Fresh | 4.48 × 10⁻³ | — |
| | Dried | 1.15 × 10⁻¹¹ | 3.84 | | Dried | 1.32 × 10⁻¹¹ | 2.42 |
| Broccoli | Fresh | 2.87 × 10⁻⁴ | — | Mango | Fresh | 1.44 × 10⁻³ | — |
| | Dried | 1.03 × 10⁻¹¹ | 3.58 | | Dried | 1.11 × 10⁻¹¹ | 3.47 |
| Beet | Fresh | 2.44 × 10⁻³ | — | Blueberry | Fresh | 9.71 × 10⁻³ | — |
| | Dried | 1.06 × 10⁻¹¹ | 4.67 | | Dried | 1.09 × 10⁻¹¹ | 2.40 |
| Potato | Fresh | 2.59 × 10⁻³ | — | Tomato | Fresh | 5.84 × 10⁻³ | — |
| | Dried | 1.33 × 10⁻¹¹ | 5.63 | | Dried | 1.15 × 10⁻¹¹ | 7.30 |
| Seaweed | Fresh | 4.22 × 10⁻² | — | Orange | Fresh | 6.66 × 10⁻³ | — |
| | Dried | 1.27 × 10⁻¹¹ | 5.04 | | Dried | 1.02 × 10⁻¹¹ | 4.40 |

| | | Conductivity | Dielectric |
|---|---|---|---|
| Bread & Cereal | | (S/m) | Constant |
| White | Fresh | 2.74 × 10⁻³ | — |
| Bread | Rolled | 1.01 × 10⁻² | — |
| | Dried | 6.76 × 10⁻¹⁰ | 12.3 |
| Rye | Fresh | 7.75 × 10⁻⁴ | — |
| Bread | Rolled | 6.73 × 10⁻³ | — |
| | Dried | 3.32 × 10⁻¹¹ | 22.5 |
| Matzoh | | 9.26 × 10⁻¹¹ | 14.8 |

TABLE 7
D₃₃ of different piezoelectric materials

| Piezoelectric materials | D33 (pC/N) |
|---|---|
| Commercial lead zirconate titanate (PZT) | 550 |
| Pure gelatin film | 0.185 |
| Broccoli/gelatin film | 4.30 |
| Cabbage/gelatin film | 0.66 |
| Cauliflower/gelatin film | 3.00 |

### ADDITIONAL CONSIDERATIONS

In the process of preparation of materials for building embodiments of electronic components, different food materials were generally not treated in the same fashion. For example, in the case of vegetables or fruits, each vegetable/fruit was cut into round slices with a thickness between 1 mm and 2 mm, inclusive, and diameter between 1 cm and 4 cm, inclusive. A household food dryer was used to dry the vegetable/fruit slices at 60° C. for 12 hours, to obtain the food substances in their dried state, i.e., dried vegetable or dried fruit. It is appreciated, however, that in some embodiments the non-dried food material can be used in some form.

In the case of carbonized cotton candy, cotton and silk, several gram quantities of Cotton candy, cotton, and silk were each annealed at 280° C. for 1 hour at a heating rate of 2° C./min, and subsequently annealed at 1000° C. for 1 hour at a heating rate of 6° C./min in an argon flow. At the end of these annealing steps, carbonized cotton candy, carbonized cotton, and carbonized silk were obtained. The carbonized cotton candy, carbonized cotton, and carbonized silk were then ground into small pieces (about 300 μm in diameter) in separate mortars.

In the case of piezoelectric composite thin films, a gelatin/broccoli film was used. Such film was produced as follows: 2 g of gelatin was weighed and sprinkled over 20 g of distilled water (i.e., 20 mL). The mixture was allowed to stand for 10 min, in order for the gelatin to fully swell, after which a gelatin solution was prepared by dissolving the swollen gelatin in approximately 100 ml distilled water with constant magnetic stirring for 30 min. The temperature of the gelatin solution was maintained at 60° C. during the mixing and stirring. 1 g of broccoli powder and 1 g of glycerol were weighed and added into gelatin solution, followed by stirring for 10 min. Gelatin/broccoli films were produced by solution casting on acrylic glass plate at 50° C. for 12 hrs.

In the case of preparation of a shadow mask, desired patterns of shadow masks were designed in AutoCAD, and transferred onto a Mylar film (0.1 mm in thickness) using a laser cutter (VLS 6.60 laser cutter, Universal Laser System, Inc.). The laser cutter cut through the Mylar film to make a shadow mask with the desired pattern.

To prepare sweet potato starch dough, powdered sugar dough, and flour powder dough: Each dough was kneaded and cut into a plate with dimensions of 3×2×0.2 cm for the mechanical test. The Instron 4411 was used to perform the compression test.

Characterization of materials, components, and devices was at least in part already addressed above. In addition, for characterization of conductivity, potentiostats (Gamry Potentiostats Reference 300) and a Multimeter (Hewlett Packard) were used for raw and dried food materials. For powdered food material, a stainless steel mold with a 1-inch diameter was used to hold the powder food material, including fresh milk powder, carbonized cotton candy, 15 carbonized cotton, carbonized silk, all-purpose flour, and sugar powder. Then 30 MPa pressure was applied on the mold to produce a condensed tablet. Two stainless steel plates were placed on two sides of a food material tablet to form a “sandwich” structure for electrical conductivity measurement, FIG. 28A, using Gamry Potentiostats Reference 3000. For a liquid food material, a plastic box with copper foils on two sides, FIG. 28B was used to hold the liquid food material. Then two copper foils were connected to the Multimeter for the resistance measurement.

For measurement of inductance, the diameter of each inductor was measured using a caliper, while the inductance was measured with precision LCR meter (Hewlett-Packard 4061A semiconducting material/component test system).

During the mechanical characterization of food materials used for structural components, a given sample was placed on the platform of the material test system (Instron 4411) for compression tests. After the initial setup, a compressive force through a pressing target was loaded and recorded. The displacement of the pressing target was also recorded until failure of the sample. The first few data were used to calculate the Young's Modulus (stress (σ)/strain (ε)) ofthe sample. The stress was calculated by dividing the force applied by the top area of the sample (force (F)/area (a)), and the strain was calculated by dividing the displacement by the thickness of the sample (displacement (Δl)/thickness (l)). Then the Young's Modulus was calculated by dividing the value of stress by the value of strain, as described above for additional compressive force, displacement, area and thickness of the sample.

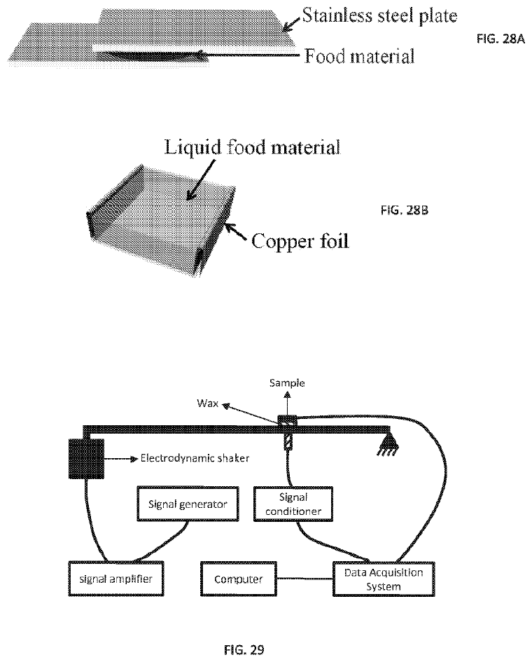

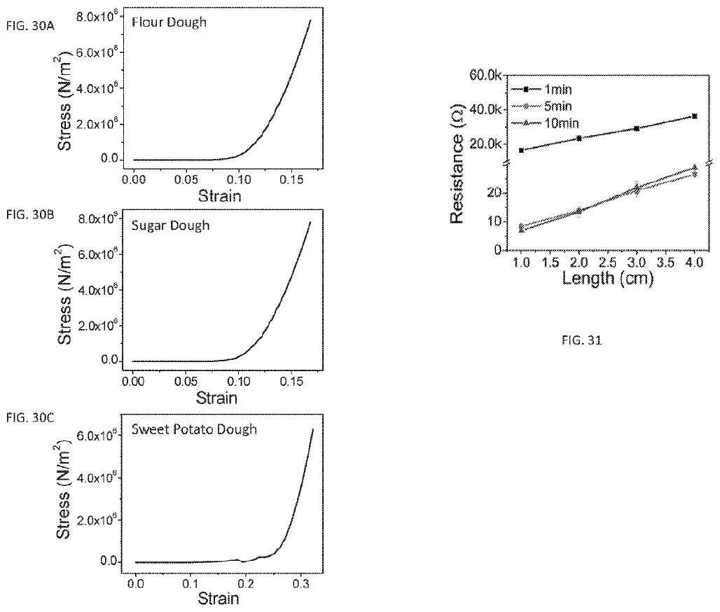

For characterization of the piezoelectric coupling coefficient, the sample's dimensions, weight, and capacitance were measured before the test. A schematic of the characterization is shown in FIG. 29. Abeam was fixed at one end, while the other end was attached to an electric shaker. The sample was attached onto the beam at the three-quarters of the distance towards the end of the shaker using wax. An accelerometer was mounted at the same location to measure the acceleration of the sample. The electric shaker was connected to its power supply and a signal generator having frequency of 50 Hz. During the vibration applied by the shaker, the inertia force was applied to the sample via F=ma, which generates a voltage, V, at the sample that was characterized by a signal analyzer. The piezoelectric coefficient (d₃₃) was assessed according to d₃₃=CV/F, where C is the capacitance of the sample (calculated separately). Coefficient d₃₁ was characterized with the use of a fatigue load frame (Bose ElectroForce Biodynamic 5160), while current was measured with a picoammeter (Keithley 6485), FIG. 4A. The samples were prepared with 8×25.4 mm Ag paint electrodes on the top and bottom surfaces. The electrodes were then extended with Cu tape to allow a proper connection with the picoammeter, FIG. 4B. A dynamic force was applied on the sample to measure periodic output current.

The Young's modulus of flour dough, sugar dough, and sweet potato dough are shown in FIGS. 30A, 30BB, and 30C, respectively. Though both sweet potato starch and wheat flour can be used as the substrate in resistors and inductors, a dough of sweet potato starch is easily shaped and tends not to fracture in the dried state, compared with regular flour. The reason is that sweet potato starch contains more starch than flour, and starch will gelatinize in the presence of water and heat. After gelatinization, starch dough will become uniform and sticky which makes it easy to be shaped into desired shapes with smooth surface. Thus, sweet potato starch can serve as a good substrate for resistors and inductors.

Embodiments of discussed electrical systems can be manufactured using processes known to one of ordinary skill in the art. These methods include, but are not limited to, screen printing, additive manufacturing, spray drying, and extrusion. The examples provide guidance on how to fabricate these electrical systems. Selected food materials may be processed via specific means to yield a range of forms. As a non-limiting example, an edible pH sensor can be fabricated using the food-based materials described above. The edible pH sensor, for example, includes edible electronic components, such as electrodes made from Au and ZnO, an antenna made of gold, and a capacitor, all mounted on an edible substrate (e.g., an edible matzoth substrate or an edible sugar-based substrate). The reaction of ZnO with either acidic or basic solutions can be used to change the capacitance between Au and ZnO electrodes, and thus the resonant frequency of the edible pH sensor. The resonant frequency of the edible pH sensor can be detected using a suitable electronic circuitry, such as a commercially available circuit made of a reader, a differential amplifier, and a spectrum analyzer. The edible pH sensor can be calibrated using the pH value of the solutions measured by a pH meter (commercially available, for example, from Hanna Instruments). During the calibration process, the edible pH sensors can be immersed in testing solutions with pH values from 1 to 14.

Fabrication of systems utilizing the described enteral electronic base materials and food kit offers a new class of materials and components that may be utilized outside of human or animal applications. Devices may be formed from these materials that may be utilized in the environment representing an additional form of biodegradable green materials. Examples here include temperature sensors, wind sensors, dust sensors, flow and drainage systems, and soil pH systems. In situations where access to expensive constituent materials an organic biodegradable's is limited food-based materials offer a practical and economic advantage.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

Overall, electronic devices and electronic components, collectively referred to as electrical systems, have been fabricated in whole or in part with food materials, processed food materials, food-like materials, and/or foodstuffs. The electronic devices and electronic components are compatible with the enteral tract; passively or actively ingestible, biodegradable and/or biocompatible, or a combination thereof. The electronic devices, electronic components, which are compatible with the enteral tract, may be passively or actively placed or otherwise localized anywhere in the GI tract, from mouth to anus. They can be placed using devices such as scopes or trochars, swallowed, via swallowing or NG tube infusion; and biocompatible, biodegradable, or a combination thereof. They may be assimilatable with contained nutritive elements and/or have compatible non-absorbed elements, such as bran or corn pericarp, which pass through the gut without digestion or assimilation and are eliminated. Elements of the systems may be partially or fully assimilated by the body or may be nutritive. The systems may be utilized for non-medical applications, such as in food packaging or in the environment where the devices fully degrade within a defined time period.

In some forms, the food materials are natural food materials such as unprocessed fruits and vegetables. In other forms the food materials may be partially or fully processed, such as ground wheat or bran from raw grain; or flour made from wheat, corn, rye, or other grains, or cooked egg or components thereof. In some forms, the electrical systems contain processed food materials and/or non-toxic levels of traditional electronic materials in order to enhance the electrical properties (e.g. electrical conductivities) of desired electronic components. In some forms, the processed food materials can be carbonized food materials, such as carbonized plant fibers such as oat or wheat bran, barley, rye, timber sources or cotton, carbonized cotton candy, or carbonized silk. In some forms, the non-toxic traditional electronic material can be a metal or metal oxide such as gold or zinc (II) oxide, or a material such as carbon or carbon nanotubes. In some forms, the electrical systems contain food-like or other natural materials, or a combination thereof, for example, polysaccharides like cellulose, sugars, proteins such as silk, gelatin, collagen or coagulated egg white (albumen), extracellular matrix, vegetable or animal oils or fats like olive, corn, and nut oils, and materials such as bone, hydroxyapatite and components thereof.

While certain implementations have been described, these implementations have been presented by way of example only, and are not intended to limit the teachings of this disclosure. Indeed, the novel methods, apparatuses and systems described herein may be embodied in a variety of other forms; furthermore, various omissions, substitutions and changes in the form of the methods, apparatuses and systems described herein may be made without departing from the spirit of this disclosure.

1. An edible electrical system comprising at least one material from a first set of materials that includes
food,
a food-like material,
processed food,
a foodstuff,
a food-based material,
a synthetic material, and
a natural material.

2. The edible electrical system according to claim 1, further comprising at least one material from a second set of materials that includes carbon, non-toxic metal, and a non-toxic non-metallic inorganic material.

3. The edible electrical system according claim 1, wherein a material from at least one of the first and second sets of materials is an electrically-conducting material, a dielectric material, or a piezoelectric material

4. The edible electrical system according to claim 1, comprising a component configured as at least one of an electrically-insulating member, a dielectric member, an electrically-conducting member.

5. The edible electrical system according claim 1, comprising a component configured as at least one of a resistor, a capacitor, an inductor, an antenna, an interconnect, a transistor, a diode, a substrate, a wire, and a printed circuit board.

6. The edible electrical system according claim 1, comprising at least one of a microphone and a pH sensor.

7. The edible electrical system according to claim 1, wherein the first set of materials includes one or more of a vegetable, fruit, bread, flour, oil, carbonized cotton candy, hard candy, cotton, carbonized cotton fiber, silk, carbonized silk, bone, tendon, gelatin, grain, sugar, active, charcoal, marshmallow, a component of an egg, and starch.

8. The edible electrical system according to claim 4, wherein a component or group of components of said edible electrical system is coated with a coating material from the first set of materials.

9. The edible electrical system according to claim 1, wherein the coating material includes at least one of a gelatin and a sugar paste.

10. The edible electrical system according to claim 8, wherein said coating material substantially encapsulates said component or groups of components.

11. (canceled)

12. (canceled)

13. The edible electrical system according to claim 1, containing a component mounted on an edible printed circuit board and, optionally, operationally connected to a microchip electronic circuitry.

14. The edible electrical system according to claim 1, containing at least two components connected directly or indirectly to electronically communicate with one another other.

15. The edible electrical system according to claim 1, wherein the first set of materials further includes at least one of a plasticizer and sorbitol.

16.-19. (canceled)

20. An implantable electrical system comprising:
at least one of i) a first material from a first set of materials that includes food, a food-like material, a foodstuff, a food-based material, a synthetic material, and a natural material, and ii) a second material from a second set of materials that includes carbon, non-toxic metal, and a non-toxic non-metallic inorganic material.

21. The implantable electrical system according to claim 20, further comprising at least one of
a) a component configured as at least one of an electrically-insulating member, a dielectric member, an electrically-conducting member, and
b) a component configured as at least one of a resistor, a capacitor, an inductor, an antenna, an interconnect, a transistor, a diode, a substrate, a wire, a printed circuit board, and a microchip.

22. The implantable system according to claim 20 and configured for being administered to a tissue orally, buccally, nasally, rectally, or vaginally.

23. The implantable electrical system according to claim 20, comprising a device selected from the group consisting of a pH sensor, a radio frequency (RF) filter, a microphones, a mechanical sensor, a temperature sensor, a pressure sensor, an analyte sensor, a microbial sensor, and a fluid-flow sensor.

24. The implantable electrical system according to claim 20, wherein a component of said system is configured to be at least one of edible, biodegradable, and ingestible.

25. An implantable electrical system comprising:
at least one of i) a first material from a first set of materials that includes food, a food-like material, a foodstuff, a food-based material, a synthetic material, and a natural material, and ii) a second material from a second set of materials that includes carbon, non-toxic metal, and a non-toxic non-metallic inorganic material,
and
at least one of a) a component configured as at least one of an electrically-insulating member, a dielectric member, an electrically-conducting member, and
b) a component or device configured as at least one of a resistor, a capacitor, an inductor, an antenna, an interconnect, a transistor, a diode, a substrate, a wire, a printed circuit board, and a microchip.

26. The implantable electrical system according to claim 25, comprising a device selected from the group consisting of a pH sensor, a radio frequency (RF) filter, a microphones, a mechanical sensor, a temperature sensor, a pressure sensor, an analyte sensor, a microbial sensor, and a fluid-flow sensor.
